(19) United States
(12) Patent Application Publication
(10) Pub. No.: US 2021/0220570 A1
(43) Pub. Date: Jul. 22, 2021

(54) NEEDLE CANNULA WITH A GRINDED POINT

(71) Applicant: Novo Nordisk A/S, Bagsvaerd (DK)

(72) Inventors: Niels Frederik Keiser-Nielsen, Nivaa (DK); Henrik Soenderskov Klint, Lyngby (DK)

(21) Appl. No.: 17/055,484

(22) PCT Filed: May 8, 2019
(86) PCT No.: PCT/EP2019/061830

§ 371 Date: Nov. 13, 2020

(30) Foreign Application Priority Data
May 17, 2018 (EP) 18172910.4

(51) Int. Cl. A61M 5/32

(52) U.S. Cl. CPC A61M 5/3286

(57) ABSTRACT

The invention relates to a needle cannula having a proximal end, a distal end and a lumen there between. The needle cannula is provided with a sharp point formed in a three-facet grinding. The first ground facet is of elliptic shape and grinded symmetrically around a central axis, and the second and third ground facet are grinded substantially symmetrically on opposite sides of the first ground facet such that the second and third ground facet converge to form the distal needle tip. The length of each of the substantially identical second and third ground facet measured along the central axis is less than half, and preferably less than one third, of the length of the first ground facet measured along the central axis.

### THE TECHNICAL FIELD OF THE INVENTION

The invention relates to a needle cannula and especially to the grinded sharp tip of a needle cannula. The invention especially relates to a needle cannula with less ability of form hooks at the sharp tip.

### DESCRIPTION OF RELATED ART

An example of an elongated tubular hollow needle cannula for injecting a liquid fluid into a subject or retracting a liquid fluid from a subject is disclosed in WO 2002/076540. In one example illustrated in FIG. 5, the disclosed needle cannula is secured in a needle hub such that a distal end extends in a distal direction and a proximal end extends in a proximal direction. The distal end is grinded to form a sharp tip to penetrate the skin of the subject to be injected and the proximal end is grinded to form an opposite tip which penetrates through a septum in a cartridge of an injection device when the injection needle is connected to the injection device. An injection needle as disclosed on FIG. 5 in WO 2002/076540 is usually referred to as a pen needle and is especially suitable for pen shaped injection device as the ones commercially used to inject insulin, GLP-1 or growth hormone. However, the needle cannula can alternatively be secured in a luer hub or directly to the injection device as also illustrated in WO 2002/076540.

The distal end of such needle cannulae which are the end of the needle cannula used to penetrate the skin of the subject is usually grinded in a three-facet or three-bevel point as disclosed in U.S. Pat. No. 2,697,438 and in WO 2018/010709.

After the needle cannula has been drawn to its correct outer and inner dimensions the individual cannulae are cut into their preferred length where after at least the distal end of each individual needle cannula is grinded. The distal end is first grinded in a first ground facet having a substantially elliptic shape grinded substantially symmetrically around the central axis.

Following the first grinding each side of the first ground facet are grinded in a second ground facet and a third ground facet by rotating the needle cannula relatively to the central axis in opposite rotational directions for each of the second ground facet and the third ground facet. The second ground facet and the third ground facet are substantially symmetrical grinded around the central axis and the needle cannula is usually also held in an angled position as the second ground facet and the third ground facet is being grinded.

The second ground facet and the third ground facet converge to form the needle point or needle tip. As seen in WO 2018/010709 and in U.S. Pat. No. 2,697,438, the closed side of the distal tip can be bended towards the central axis to better protect the lumen of the needle cannula from obtaining particles from the septum as the needle cannula penetrates the septum of the injection pen.

In the needle cannulae disclosed in the prior art, the length of the second ground facet and the third ground facet are relatively long which makes the sharp tip very thin and thus vulnerable to form hooks. If the sharp tip of the needle cannula is exposed to an axial force, a very long and thin needle tip has a tendency to curl and form a hook. Such exposure to an axial force can happen if the user accidentally drops the injection device with the needle cannula onto a hard surface.

A needle cannula with a hook at the sharp tip operates somewhat like a fishing hook when the user withdraws the sharp tip from the skin following an injection. This is obviously very discomforting.

Further, in the concept disclosed in WO 2014/064100 and in WO 2015/062845, the needle cannula is permanently mounted on the injection device and is used for multiple injections throughout the lifetime of the injection device. The general concept is thus that the lifetime of the needle cannula follows the lifetime of the injection device. When the injection device is an injection pen prefilled with e.g. 300 International Units of insulin as much as 30 or even more injections can be performed using the same needle cannula.

For each injection the distal tip of the needle cannula has to pass both through a septum in the cleaning chamber and through the skin of the user. The sharp tip of the needle cannula is thus exposed to a relatively high axial force several times and the wear on the distal tip of the needle cannula is henceforth substantial. At the same time very thin needle cannulae as reflected in the below table are preferred by many people. Such combination of extremely thin needle cannulae exposed to a higher than normal axial force also exposes the sharp needle tip to curling and hook formation.

The below table shows the needle dimensions from ISO 9626 which defines the inner and outer diameter of tubular needle cannulae. For self-injection performed by people suffering from a chronical dieses such as diabetes or growth disorder needle cannulae in the range G28 to G34 seem to be preferred.

TABLE 1
ISO 9626: Second Edition 2016

| | | | | Inner | Inner |
|---|---|---|---|---|---|
| | | Outer | Outer | diameter | diameter |
| | Designated | diameter | diameter | (lumen) | (lumen) |
| Gauge | metric size | min | Max. | Min. | Max. |
| 34 | 0.18 | 0.178 | 0.191 | 0.064 | 0.105 |
| 33 | 0.20 | 0.203 | 0.216 | 0.089 | 0.125 |
| 32 | 0.23 | 0.229 | 0.241 | 0.089 | 0.146 |
| 31 | 0.25 | 0.254 | 0.267 | 0.114 | 0.176 |
| 30 | 0.30 | 0.298 | 0.320 | 0.133 | 0.240 |
| 29 | 0.33 | 0.324 | 0.351 | 0.133 | 0.265 |
| 28 | 0.36 | 0.349 | 0.370 | 0.133 | 0.190 |

### DESCRIPTION OF THE INVENTION

It is an object of the present invention to provide a very thin needle cannula having a sharp tip that is more resistant to hook formation.

Accordingly, in one aspect of the present invention, a thin tubular needle cannula having a proximal end and a distal end and a lumen along a centre axis is provided.

The distal end of this needle cannula doing the actual penetration of the skin of the user is formed in a sharp point which comprises:
a first ground facet with a substantially elliptic shape grinded substantially symmetrically around the centre axis, and
a substantially identical second and a third ground facet grinded substantially symmetrically on opposite sides of the first ground facet and which second and third ground facet converge to form a distal needle tip.

According to the present invention, the length of each of the second and third ground facet measured along the central axis is less than half the length of the first ground facet also measured along the central axis. The ratio between the second and third ground facet and the first ground facet is thus less than 0.5.

Various tests indicate that a relatively short length on the second and third ground facet relatively to the length of the first ground facet makes the needle tip less vulnerable to hook formation should the needle tip be exposed to an axial force.

Following this, the length of each of the second and third ground facet measured along the central axis should be less than a third of the length of the first ground facet measured along the central axis such that the ratio is less than 0.33.

The preferred length of the first ground facet measured along the central axis is in the range of 0.9 mm to 1.35 mm and preferably around 1,125 mm+1-0.15 mm.

The preferred length of the second and the third ground facet measured along the central axis is in the range of 0.15 mm to 0.6 mm and preferably around 0,196 mm to 0.56 mm.

A needle point following the above has demonstrated to be very resistant to hook formation. Many commercially needle cannulae starts to curl and form a hook when exposed to an axial force in the area of 1 to 2 Newton. However, with the needle point being grinded as above a substantially higher axial force needs to be applied before the needle cannula starts to curl. In the examples described herein, an axial force of 8 Newton and 12 Newton is required to make a hook thus making needle cannulae according to the present invention very resistant to hook formation.

When the distal needle tip is exposed to an axial force sufficiently high to form a hook such hook usually bends outwards from the outer surface of the needle cannula. This is very unfortunate since such hook causes a higher degree of pain on the user both during penetration of the skin but in particular upon removal from the skin as the shape of the hook is somewhat like a fishing hook.

However, when the distal needle tip is bended the hook formed at the distal needle tip has a tendency to point in the opposite direction. This means that the hook is curved towards the centre line and towards the opposite outer surface. During injection, the hook is thus deflected within the outer dimensions of the needle contour which significantly lowers the pain felt during injection when compared to a nonbended needle cannula having an outwardly pointing hook.

Thus, a needle cannula with the above grinding and a bended distal needle tip is more resistant to hook formation, and should a hook form, this hook points inwardly and lies within the outer contour of the needle cannula.

In a preferred embodiment, a bending part or area of the distal end of the needle cannula is bended whereas the actual distal tip itself is not actively bended. This bending area preferably starts 0.1 to 0.2 mm proximally from the distal needle point and the distal end is bended such that the radial position of the distal needle tip is positioned a radial distance from the outer surface of the needle cannula.

In one embodiment, this radial distance is such that distal needle point when bended lies within the clearance of the hollow lumen and in a second embodiment, the radial distance is such that the distal needle point lies substantially on the central axis of the needle cannula. The latter position provides convincing evidence that the hook actually points inwardly when deflected.

When grinding the first ground facet a planar surface following the first ground facet (5) forms an angle α with the central axis of the needle cannula in the range of 10° to 15°. The angle α is preferably in the area of 11.5° to 13.8°.

When grinding the second and the third ground facet, the needle cannula is rotated around the centre axis an angle γ which is in the range of 120° to 160° and most preferably around 140°+/−10°. As the needle cannula is rotated to the two outer position for grinding the second and the third ground facet, the needle cannula is also tilted an angle β which is in the range of 25° to 35° and preferably around 30+/−3 . . . .

The needle cannula according to the present invention is preferably a G28, G29, G30, G31, G32, G33 or G34 needle following the ISO 9626 standard, however, it is noted that any of the above needle cannulae can be made conical at the distal end as e.g. described in WO 2002/076540.

Different examples of the dimensions of the distal point of the needle cannula according to the present invention is provided in example 1 and in example 2.

### Definitions

An “injection pen” is typically an injection apparatus having an oblong or elongated shape somewhat like a pen for writing. Although such pens usually have a tubular cross-section, they could easily have a different cross-section such as triangular, rectangular or square or any variation around these geometries.

The term “Needle Cannula” is used to describe the actual conduit performing the penetration of the skin during injection. A needle cannula is usually made from a metallic material such as e.g. stainless steel and preferably connected to a hub to thereby form a complete “injection needle” also often referred to as a “needle assembly”. A needle cannula could however also be made from a polymeric material or a glass material. The hub also carries the connecting means for connecting the needle assembly to an injection apparatus and is usually moulded from a suitable thermoplastic material.

As used herein, the term “drug” is meant to encompass any drug-containing flowable medicine capable of being passed through a delivery means such as a hollow needle cannula in a controlled manner, such as a liquid, solution, gel or fine suspension. Representative drugs includes pharmaceuticals such as peptides, proteins (e.g. insulin, insulin analogues and C-peptide), and hormones, biologically derived or active agents, hormonal and gene based agents, nutritional formulas and other substances in both solid (dispensed) or liquid form.

“Cartridge” is the term used to describe the container actually containing the drug. Cartridges are usually made from glass but could also be moulded from any suitable polymer. A cartridge or ampoule is preferably sealed at one end by a pierceable membrane referred to as the “septum” which can be pierced e.g. by the non-patient end of a needle cannula. Such septum is usually self-sealing which means that the opening created during penetration seals automatically by the inherent resiliency once the needle cannula is removed from the septum. The opposite end is typically closed by a plunger or piston made from rubber or a suitable polymer. The plunger or piston can be slidable moved inside the cartridge. The space between the pierceable membrane and the movable plunger holds the drug which is pressed out as the plunger decreased the volume of the space holding the drug. However, any kind of container—rigid or flexible—can be used to contain the drug.

Since a cartridge usually has a narrower distal neck portion into which the plunger cannot be moved not all of the liquid drug contained inside the cartridge can actually be expelled. The term “initial quantum” or “substantially used” therefore refers to the injectable content contained in the cartridge and thus not necessarily to the entire content.

By the term “Pre-filled” injection device is meant an injection device in which the cartridge containing the liquid drug is permanently embedded in the injection device such that it cannot be removed without permanent destruction of the injection device. Once the pre-filled amount of liquid drug in the cartridge is used, the user normally discards the entire injection device. This is in opposition to a “Durable” injection device in which the user can himself change the cartridge containing the liquid drug whenever it is empty. Pre-filled injection devices are usually sold in packages containing more than one injection device whereas durable injection devices are usually sold one at a time. When using pre-filled injection devices an average user might require as many as 50 to 100 injection devices per year whereas when using durable injection devices one single injection device could last for several years, however, the average user would require 50 to 100 new cartridges per year.

Using the term “Automatic” in conjunction with injection device means that, the injection device is able to perform the injection without the user of the injection device delivering the force needed to expel the drug during dosing. The force is typically delivered—automatically—by an electric motor or by a spring drive. The spring for the spring drive is usually strained by the user during dose setting, however, such springs are usually prestrained in order to avoid problems of delivering very small doses. Alternatively, the spring can be fully preloaded by the manufacturer with a preload sufficient to empty the entire drug cartridge though a number of doses. Typically, the user activates a latch mechanism provided either on the surface of the housing or at the proximal end of the injection device to release—fully or partially—the force accumulated in the spring when carrying out the injection.

The term “Permanently connected” or “permanently embedded” as used in this description is intended to mean that the parts, which in this application is embodied as a cartridge permanently embedded in the housing, requires the use of tools in order to be separated and should the parts be separated it would permanently damage at least one of the parts.

All references, including publications, patent applications, and patents, cited herein are incorporated by reference in their entirety and to the same extent as if each reference were individually and specifically indicated to be incorporated by reference and were set forth in its entirety herein.

All headings and sub-headings are used herein for convenience only and should not be constructed as limiting the invention in any way.

The use of any and all examples, or exemplary language (e.g. such as) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

### BRIEF DESCRIPTION OF THE DRAWINGS

The invention will be explained more fully below in connection with a preferred embodiment and with reference to the drawings in which:

FIG. 1 show a side view of the needle cannula.

FIG. 2A show a top view of the needle cannula rotated 90° in relation to FIG. 1.

FIG. 2B show an enlarged section of the distal end of the needle cannula in FIG. 2A.

FIG. 3 show an end view of the needle cannula together with the grinding process.

FIG. 4 show an example of a hook formed at the sharp tip of a needle cannula.

FIG. 5 show a side view of the needle cannula being bended.

The figures are schematic and simplified for clarity, and they just show details, which are essential to the understanding of the invention, while other details are left out. Throughout, the same reference numerals are used for identical or corresponding parts.

### DETAILED DESCRIPTION OF EMBODIMENT

When in the following terms as “upper” and “lower”, “right” and “left”, “horizontal” and “vertical”, “clockwise” and “counter clockwise” or similar relative expressions are used, these only refer to the appended figures and not to an actual situation of use. The shown figures are schematic representations for which reason the configuration of the different structures as well as there relative dimensions are intended to serve illustrative purposes only.

In that context it may be convenient to define that the term “distal end” in the appended figures is meant to refer to the end of the needle cannula doing the actual penetration of the skin of a user, whereas the term “proximal end” is meant to refer to the opposite end. Distal and proximal is meant to be along an axial orientation extending along the central axis (X) of the needle cannula as also disclosed in FIG. 2A.

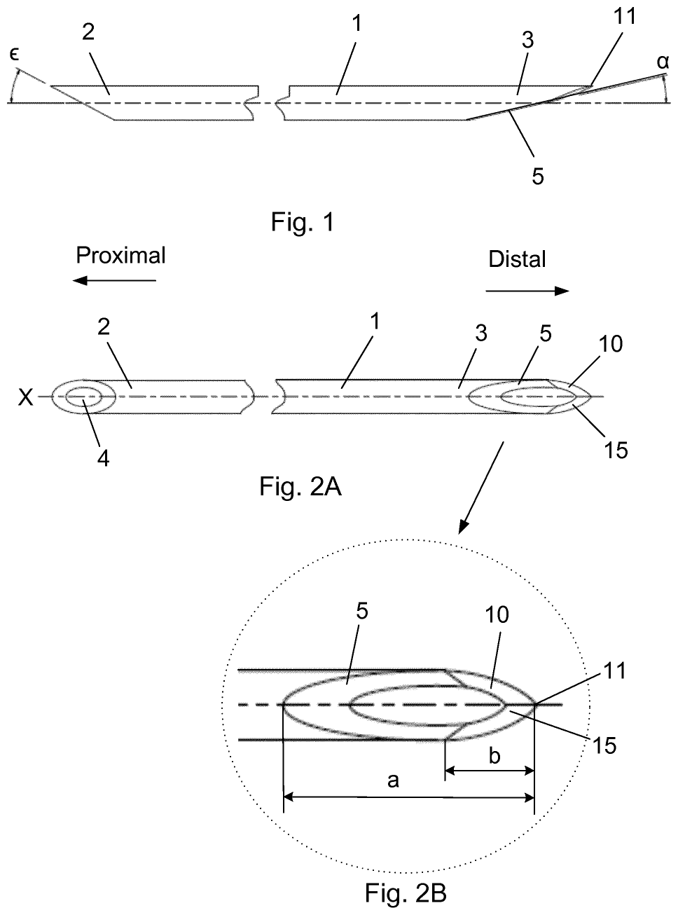

FIG. 1 discloses the needle cannula 1 which has a proximal end 2 and a distal end 3. The distal end 3 is the end that does the actual penetration of the skin of a user during injection. A hollow lumen 4 provides the flow way for the liquid drug to be injected.

The needle cannula 1 is preferably secured in a needle hub as e.g. shown in WO 2002/076540. In such example, the proximal end 2 of the needle cannula 1 penetrates through a septum in a cartridge when the injection needle is connected to an injection device.

The distal end 3 of the needle cannula 1 is grinded in a 3-facet grind. During grinding the individual needle cannula 1 is held in a fixture and the distal end 3 is grinded in a first ground facet 5. This first ground facet 5 has an elliptic shape and is substantially symmetrically placed around the central axis X as disclosed in FIG. 2A.

The angle α appearing between a planar surface following the first ground facet 5 and the central axis X is preferably between 11° to 14° and most preferably 12.5°+/−1°. The length of the first ground facet 5 is indicated as “a” in FIG. 2B and is preferably 0.9 mm to 1.35 mm and most preferably 1.125+/−0.2 mm.

The proximal end 2 of the needle cannula 1 is grinded in a 1-facet grind. The angle c used for the grinding at the proximal end is preferably 20° to 36° and most preferably 28°+/−4°. When the needle cannula 1 is used for a pen needle, the grinding at the proximal needs only to be sufficient for the proximal end 2 to penetrate through the septum of the cartridge containing the liquid drug. However any kind a grinding can eb applied to the proximal end 2 of the needle cannula 1.

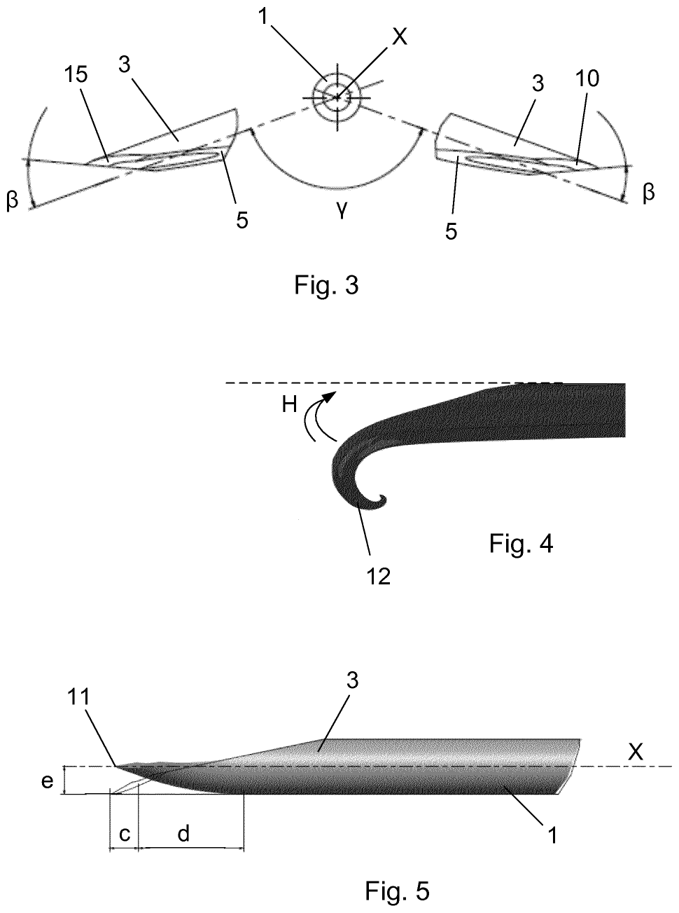

Following the grinding of the first ground facet 5, the individual needle cannula 1 is rotated around the central axis X as disclosed in FIG. 3 in order have the two sides of the first ground facet 5 grinded. The needle cannula 1 is first rotated in one rotational direction and thereafter in the opposite rotational direction. The total angle γ of rotation is 140+/−10. When rotated each side of the first ground facet 5 is grinded in a second ground surface 10 and a third ground facet 15 as illustrated in FIG. 2A-B. These two ground facets 10, 15 are grinded substantially symmetrically around the central axis X and converge into a distal needle tip 11 at the most distal end 3 of the needle cannula 1.

When the needle cannula 1 has been rotated approximately 70° to each rotational side, the second ground facet 10 and the third ground facet 15 are grinded at an angle β being between 25° to 35° and most preferably 30°+/−3°.

The axial length of the second ground facet 10 and the third ground facet 15 is indicated as “b” in FIG. 2B and is preferably less than half of the length “a” of the first ground facet 5. More preferably the length b is less than a third of the length a of the first ground facet 5. The ratio between “b” and “a” should henceforth be less than 0.5 and preferably less than 0.33. In numbers the preferred length of b is 0.196 to 0.56 mm.

FIG. 4 shows an example of a hook 12 formed at the distal needle tip 11 of a needle cannula 1. If the distal needle tip 11 is exposed to an axial force, the distal needle tip 11 will curl and form a hook 12 pointing away from centre axis (X) of the needle cannula 1. Such hook 12 would have the shape of a fishing hook. When the user then retracts the distal needle tip 11 from the skin this hook 12 will cause extra damage to the tissue and obviously also provide a less comfortable feeling.

For many injection needles commercially available it can be measured that such hook 12 is formed when the axial force on the distal needle tip 11 is higher than approximately 1 to 2 newton. However, experiments have demonstrated that if the length of the second ground facet 10 and the third ground facet 15 is made sufficiently short the force needed to form a hook 12 will increase dramatically.

### Examples of the Invention

Two identical G30 needle cannulae 1 have been grinded in a three-facet grind with the following dimensions;

### Example 1

α=12.8°+/−1°
β=30°+/−3°
γ=140°+/−10°
a=1.125 mm+/−0.15 mm
b=0.41 mm+/−0.15 mm
b/a=0.36
Hook resistance=8 Newton

### Example 2

α=12.5°+/−1°
β=30°+/−3°
γ=140°+/−10°
a=1.125 mm+/−0.15 mm
b=0.296 mm+/−0.1 mm
b/a=0.26
Hook resistance=12 Newton

Following the grinding of the three-facet grinds, the force required to form a hook 12 was measured. In the first example, a hook 12 was formed when the force reached 8 Newton, and in the second example, the force required to form a hook was 12 Newton.

In both examples, the force needed to form a hook at the distal needle tip 11 was substantially higher than in the commercially available needle cannulae. The needle cannulae 1 according to the invention were thus more hook resistant when compared to commercially available needle cannulae.

FIG. 5 show an example wherein the distal needle tip 11 has been bended inwards toward the centre axis X. As seen in FIG. 5, the distal end 3 is provided with a bending area “d” which is located a proximal distance “c” from the distal needle tip 11.

In a preferred embodiment, the bending area “d” has a length on 0.55 mm+/−0.5 mm and is stretching in the proximal direction from a distance c of 0.15 mm+/−0.05 mm from the distal needle tip 11.

The actual bending of the distal end 3 is made by bending the bending area “d” without actively bending the outer distal distance “c”. The outer contour of the needle cannula 1 prior to bending is indicated in FIG. 5.

The distal needle tip 11 is preferably bended to a position in a radial distance “e” from the outer surface of the needle cannula 1. This radial distance “e” is preferably such that the distal needle tip 11 lies within the clearance of the lumen 4 of the needle cannula 1. Most preferably is the distal needle tip 11 bended to a position wherein it follows the central axis X of the needle cannula 1 as disclosed in FIG. 5. The distance e would thus be half the outer diameter of the needle cannula 1.

In one example the needle cannula 1 could be a G30 cannula with an outer diameter on 0.31 mm+/−0.01 mm and an inner diameter on 0,175 mm+/−0.01 mm and wherein “e” is 0,155 mm+/−0.1 mm.

When the distal needle tip 11 of a needle cannula 1 without bending is exposed to an axial force and forms a hook 12, this hook 12 will point in a direction outwardly from the outer surface of the needle cannula 1 and away from the lumen 4 as disclosed in FIG. 4. However, when the needle cannula 1 is bended at the distal end 3, as suggested herein, the hook 12 will form to the opposite side, i.e. towards the central axis X and the opposite surface of the needle cannula 1 as indicated by the arrow H in FIG. 4. Once the hook 12 lies within the contour of the needle cannula 1 it becomes more comforting for the user to remove the needle cannula 1 from the skin as the damage to the tissue and the following pain is greatly reduced.

Some preferred embodiments have been shown in the foregoing, but it should be stressed that the invention is not limited to these, but may be embodied in other ways within the subject matter defined in the following claims.

1. A needle cannula having a proximal end and a distal end and a lumen there between and wherein the distal end (3) is provided with a sharp point,
the sharp point comprising:
a first ground facet with a substantially elliptic shape grinded substantially symmetrically around a central axis (X),
a second ground facet and a third ground facet grinded substantially symmetrically on opposite sides of the first ground facet and which second ground facet and third ground facet converge to form a distal needle tip,
wherein,
the length (b) of each of the second ground facet and of the third ground facet measured along the central axis (X) is less than half the length (a) of the first ground facet measured along the central axis (X).

2. The needle cannula according to claim 1, wherein the length (b) of each of the second ground facet and of the third ground facet measured along the central axis (X) is less than a third of the length (a) of the first ground facet measured along the central axis (X).

3. The needle cannula according to claim 1, wherein the length (a) of the first ground facet measured along the central axis (X) is in the range of 0.9 mm to 1.35 mm.

4. The needle cannula according to claim 1, wherein the length (b) of the second ground facet and of the third ground facet measured along the central axis (X) is in the range of 0.15 to 0.6 mm.

5. The needle cannula according to claim 1, wherein the distal end of the needle cannula is bended.

6. The needle cannula according to claim 5, wherein a bending area (d) of the distal end starting 0.1 mm to 0.2 mm proximally from the distal needle tip is bended.

7. The needle cannula according to claim 5, wherein the needle tip is bended to a position a radial distance (e) from the outer surface of the needle cannula.

8. The needle cannula according to claim 5, wherein the distal needle tip when bended lies within the clearance of the hollow lumen.

9. The needle cannula according to claim 5, wherein the distal needle tip when bended lies substantially on the central axis (X).

10. The needle cannula according to claim 1, wherein a planar surface following the first ground facet forms an angle α with the central axis (X) of the needle cannula and wherein the angle α is in the range of 10° to 15°.

11. The needle cannula according to claim 1, wherein the needle cannula is rotated an angle γ between grinding of the second ground facet and the third ground facet and wherein the angle γ is in the range of 120° to 160°.

12. The needle cannula according to claim 1, wherein the second ground facet and the third ground facet forms an angle β with the central axis (X) of the needle cannula and wherein the angle β is in the range of 25° to 35°.

13. The needle cannula according to claim 1, wherein the needle cannula is a G28, G29, G30, G31, G32, G33 or G34 needle.

14. The needle cannula with a distal sharp point according to claim 1.
